(19) United States
(12) Plant Patent Application Publication
(10) Pub. No.: US 2013/0074235 P1
(43) Pub. Date: Mar. 21, 2013

(54) Ornithogalum plant named 'Lourdes'

(50) ;

(71) Applicant: Rodolfo Valdoz Bautista, Half Moon Bay, CA (US)

(72) Inventor: Rodolfo Valdoz Bautista, Half Moon Bay, CA (US)

(21) Appl. No.: 13/200,051

(22) Filed: Sep. 16, 2011

(51) Int. Cl. A01H 5/00

(52) U.S. Cl. PLT/313

(57) ABSTRACT

A new and distinct cultivar of Ornithogalum dubium named ‘Lourdes’ originated as a controlled hybridization that started with the crossing of the commercial cultivar Ornithogalum thyrsoides Jacq. ‘Bethlehem’ and an unnamed Ornithogalum dubium cultivar. Progeny of this cross were then crossed back to the Ornithogalum dubium until the present variety was selected. The cultivar ‘Lourdes’ has R.H.S. 25A (orange group) tepals at maturity, erect and strong racemes which resist bending under low light conditions, a long blooming habit and broad, channeled leaves.

### BOTANICAL CLASSIFICATION

Ornithogalum dubium ‘Lourdes’

### VARIETY DENOMINATION

‘Lourdes’

### BACKGROUND OF THE INVENTION

This invention relates to a new and distinct cultivar of the Lilaceae family. The botanical name of the plant is Ornithogalum dubium ‘Lourdes’.

The new cultivar is the result of controlled crossing that started with the crossing of the commercial variety Ornithogalum thyrsoides Jasq. ‘Bethlehem’ (U.S. Plant Pat. No. 21,427) and an unnamed Ornithogalum dubium. The unnamed Ornithogalum dubium was the seed parent in the original cross. The progeny of this cross were crossed back to the dubium parent, and this crossing back to the original dubium parent was continued until the new variety was selected for development. The patented ‘Bethlehem’ variety originated as a seedling from a controlled cross between the commercial variety Ornithogalum thyrsoides ‘Snowflake’ and an unnamed Ornithogalum thyrsoides. ‘Snowflake’ is the progeny of the commercial variety ‘Bok Bay’.

The new variety ‘Lourdes’ is differentiated from other commercial varieties known to the inventor as shown in the table below.

TABLE 1

| | | | ‘Chesapeake | |
|---|---|---|---|---|
| | New Variety | ‘Bethlehem’ | Snowflake’ | ‘Orangezicht’ |
| | ‘Lourdes’ | USPP 21,427 | USPP 13,200 | USPP 12,226 |
| Rachis/Raceme | 40 to 50 cm | 43 cm | 20-30 cm | 26 cm |
| height | | | | |
| Rachis color | Varies between | R.H.S. 146A | Not reported | Not reported |
| | R.H.S. 146B, | | | |
| | R.H.S. 147B, | | | |
| | or | | | |
| | R.H.S. 145D | | | |
| Reflexed leaves | No | Yes | Not reported | Leaves lay flat |
| | | | | on ground |
| Leaf color | R.H.S. 137A | R.H.S. 146A | R.H.S. 143B | Upper surface |
| | | | | R.H.S. 137A |
| | | | | or 147A. |
| Floret diameter | 30 mm | 70 mm | 50-60 mm | 50-60 mm |
| Tepal height | 30 mm | 36 mm | 20-25 mm | 25-27 mm |
| Tepal Color | R.H.S. 25A | R.H.S. 155C | R.H.S. 155A-D | Varies |
| | | | | between |
| | | | | R.H.S. 25A, |
| | | | | R.H.S. 25D, or |
| | | | | R.H.S. 28A |
| Tepal Spot | R.H.S. 152B | R.H.S. 152A | R.H.S. 147A | R.H.S. 200B |
| Color | | | | |

The new variety is suitable for commercial sale as a potted plant and has the following unique combination of desirable characteristics: florets on the raceme will bloom for 10-12 weeks, following the blooming of the first floret; broad, channeled leaves; strong stems; and under low-light conditions, typical in homes, the raceme and stem will remain compact and not droop.

The new cultivar ‘Lourdes’ has been asexually reproduced through three generations under controlled environmental conditions at a nursery in Half Moon Bay, Calif. under the direction of the inventor with its distinguishing characteristics remaining stable.

Asexual reproduction was first accomplished when vegetative cuttings were taken from the initially selected plant. Examination of asexually reproduced, successive generations grown in a nursery at Half Moon Bay, Calif. show that the combination of characteristics as herein disclosed for ‘Lourdes’ remains firmly fixed through three generations.

### DESCRIPTION OF THE DRAWINGS

The accompanying drawings consist of color photographs that show the typical plant form, including the inflorescence, foliage, and sepals.

FIG. 1 is a view of the new variety showing its form.

FIG. 2 is a top view of two inflorescences of the new variety.

FIG. 3 is a close-up view of the base of the plant of the new variety.

FIG. 4 is a close-up view of the top of the inflorescence of the new variety.

FIG. 5 is a top view of a number of inflorescences of the new variety.

FIG. 6 is a close-up view of several new florets of the new variety.

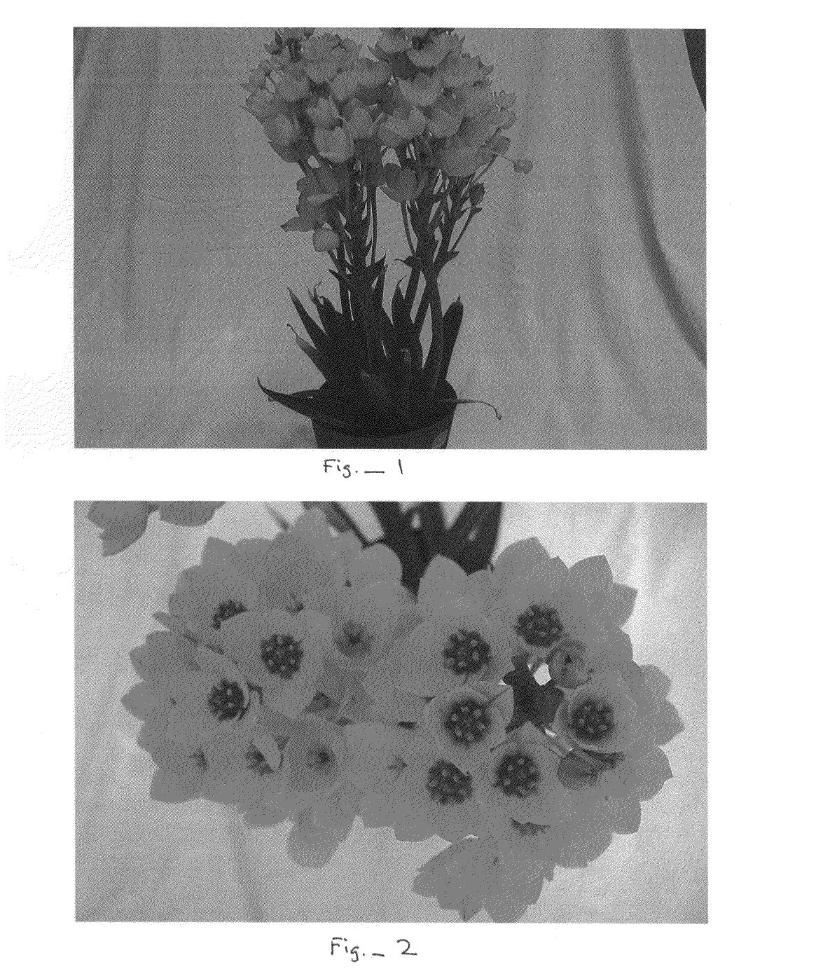

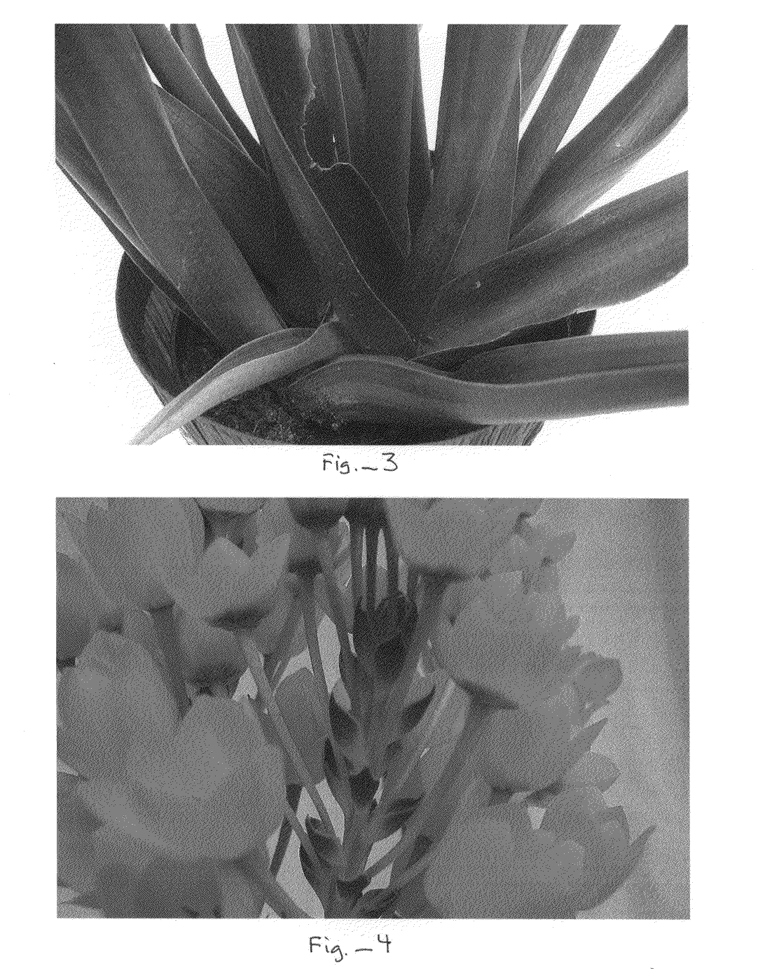

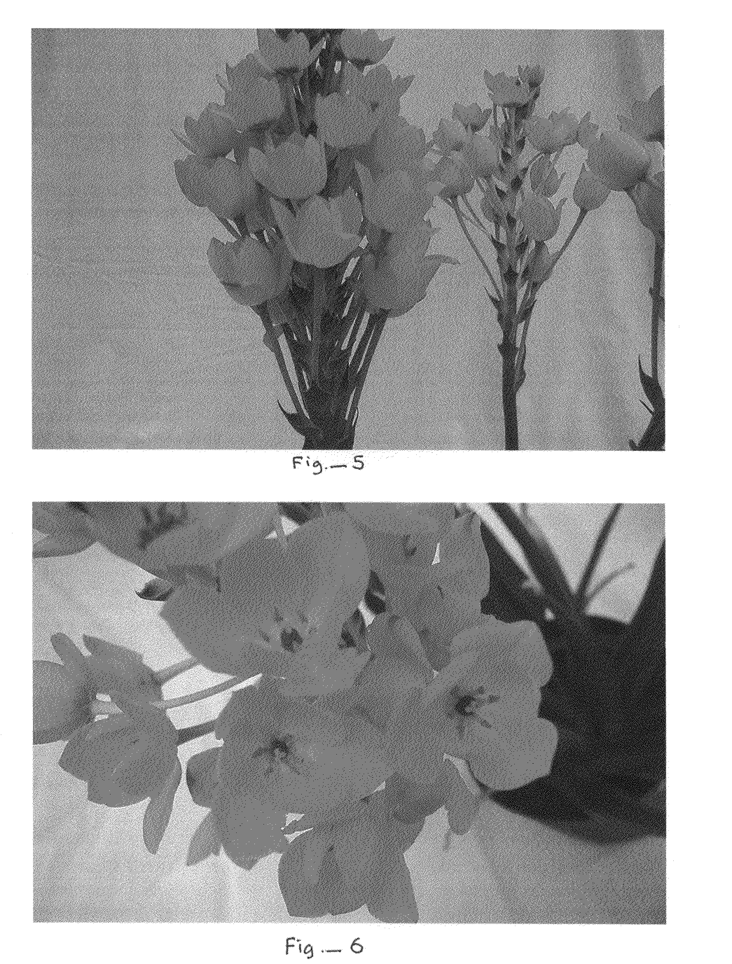

### DESCRIPTION OF THE NEW PLANT

The plants shown in the figures were grown in 6″ pots and were approximately 11 months old. The plants were started in a greenhouse from leaf cuttings.

‘Lourdes’ has not been observed under all possible environmental conditions. The phenotype may vary significantly with variations in environment such as temperature, light intensity and day length. Color determinations were made with The Royal Horticultural Society (R.H.S.) Colour Chart.
The plant:
   Origin.—Controlled cross that started with the crossing of the commercial variety Ornithogalum thyrsoides Jasq. ‘Bethlehem’ (U.S. Plant Pat. No. 21,427) and an unnamed Ornithogalum dubium. The progeny of this cross were crossed back to the dubium parent, and this crossing back of progeny to the dubium parent was repeated until the new variety was selected for development.
   Form.—Spreading basal leaves with upright inflorescences on tall rachis. A typical plant with a mature inflorescence that is ready for sale at ten months is approximately 40 cm high when grown in a 6″ pot with appropriate soil amendments. Plants shown are 50 cm high.
   Growth.—Upright rachis with upright leaves. Inflorescence is large and showy.
   Rachis.—The rachis is upright and glaucous. Its color varies between R.H.S. 146 B (yellow-green group) when younger, R.H.S. 147 B (yellow-green group), and R.H.S. 145 D (yellow-green group) when past maturity of the inflorescence. Diameter: 9 mm. The length of the rachis to the first floret and bract pair is variable and ranges from 15 cm to 25 cm. Rachis or stem at its base is 15 mm in diameter.
   Foliage.—Usually three to five leaves surround each rachis on a mature plant ready for commercial sale. Leaves are whorled at base of stem. Shape of leaf. — Fleshy, channel-shaped, lanceolate. Margins are entire. Apex is acute. Texture. — Glaucescent. Color. — Mature leaves are R.H.S. 137 A (green group) on both abaxial and adaxial surfaces. Large leaves are 23 cm long and 5 cm wide.
Inflorescence:
   Form.—Corymbose racemes. Indeterminate. Approximately 50 or more individual florets per inflorescence on pedicels that elongate as the florets opens, and on average are as long as 80 mm for mature florets. Apical buds on developed infloresences are predominately green — R.H.S. 146 B (yellow-green group), 1.2-1.5 cm tall and have diameter of 8 to 10 mm. The rachis for the inflorescence is strong and upright. The pedicels are glabrous and similar in color to the rachis at their point of attachment.
   Size of inflorescence.—Large. A typical inflorescence at this age, as shown in the figures can be 25 cm high.
   Shape.—Basal florets opening first with pedicels that elongate that give the raceme a corymbose appearance as the inflorescence ages.
   Appearance.—Showy.
Florets:
   General.—The florets at the base of the inflorescence open first, and open more and more as the inflorescence ages. Florets are perfect and complete. Perianth is not keeled. Each floret is associated with a bract that can be as high as 30 mm and is same color as leaves — R.H.S. 137 B (green group). The bracts are lanecolate with narrowly acute apexes and relatively wide bases. Perianth: There are 6 ovate tepals that are spotted at their base on both their abaxial and adaxial surfaces — R.H.S. 152 B (yellow-green group), but are predominately R.H.S. 25 A (orange group) on their abaxial and adaxial surfaces. Floret diameter: 30 mm. Tepals of mature florets are typically 30 mm high, 18 mm wide at their widest point, 4 mm wide at their attachment, and the spotted base is approximately 3 mm high and extends the width of the tepal at the base. Apex shape of the tepal is abruptly acuminate.
   Stamens.—6 stamens (two sets of 3). Pollen is R.H.S. 14 A (yellow-orange group). Plant produces abundant pollen. Filament is wide. Filament is 3 mm wide at base and 4 mm high. Anther is 3 mm high and basally attached. Filament is R.H.S. 155 A (white group). Anther is R.H.S. 146 B (yellow-green group) before opening to reveal pollen.
   Stigma.—Generally, a three-pronged stigma on one pistil per floret. The style is 2 mm long. The stigma is approximately 2 mm long.
   Ovary.—The ovary is three-celled with axile placentae. The ovary is R.H.S. 143 A (green group). The ovary is 6 mm high and has a diameter of 4 mm.
   Fragrance.—Florets product a very mild citrus-like fragrance, if any.
   Disease resistance.—The plant is not known to have any particular resistance to pests or plant diseases.
   Fruit.—None.

I claim:

1. A new and distinct Ornithogalum plant named ‘Lourdes’ substantially as herein shown and described.
